(19) United States
(12) Patent Application Publication
(10) Pub. No.: US 2025/0363310 A1
(43) Pub. Date: Nov. 27, 2025

(54) TECHNIQUES FOR TRAINING AND VALIDATING AN OPTIMIZED MACHINE LEARNING MODEL

(71) Applicant: LAYER HEALTH, INC., Brookline, MA (US)

(72) Inventors: David Alexander Sontag, Brookline, MA (US); Monica Nayan Agrawal, Boston, MA (US); Divya Gopinath, Millwood, NY (US); Steven Horng, Boston, MA (US); Luke Murray, Brooklyn, NY (US)

(21) Appl. No.: 18/674,211

(22) Filed: May 24, 2024

(51) Int. Cl. G06F 40/40; G06F 18/2415; G06F 40/20

(52) U.S. Cl. CPC G06F 40/40; G06F 18/2415; G06F 40/20

(57) ABSTRACT

A method of responding to questions about documents by a computing system includes: (a) initially sending queries to a large language model (LLM), each query including a request for any snippets within a corresponding document that provide evidence in response to a question, the LLM returning a respective response to each query; (b) in response to a heuristic based on the responses returned by the LLM indicating a readiness for optimization, training a new model on the queries and respective responses, the new model being more computationally efficient than the LLM; (c) performing a validation operation on additional responses returned by the new model; and (d) in response to the validation operation indicating that the new model has satisfied a condition, sending future queries to the new model instead of the LLM.

### BACKGROUND

Medical providers take detailed clinical notes about their interactions with patients. There is a wealth of health information embedded within these clinical notes. In order to extract this information in a useful way, trained health professionals may read through the clinical notes (sometimes referred to as “chart review”) and enter data into structured forms or databases, allowing the medical information to be analyzed in bulk.

Another way to extract useful information from clinical notes involves performing keyword searches, and having trained health professionals review the search results to reduce the amount of reading required.

### SUMMARY

The above conventional techniques for extracting useful information from clinical notes have drawbacks. Having trained health professionals read through all the clinical notes is tedious, time-consuming, and prone to user error. Performing a preliminary search may reduce the time to an extent, but it is still time-consuming, and it also adds an additional possibility of missing information that is not flagged by search. Developing machine learning models for each specific information element is also time-consuming, as it requires first having health professionals review a large number of clinical notes to label data to obtain the ground-truth needed for machine learning.

Thus, one approach has been to use a large language model (LLM)-based system to perform queries on clinical notes based on natural language questions posed by users to generate structured results. In some systems, a first query is generated from the user question to request snippets of evidence within a set of clinical notes (or any other document) that are relevant to the user's question from a first LLM; then, the snippets returned from the first LLM are used to generate a second query requesting an answer to the original question based on the snippets, the second query being fed into a second LLM. In some systems, the second query may include structural information, additional context from the source documents, and/or a set of examples to guide the second LLM towards the right answer.

Although these systems are effective, the use of LLMs can be expensive and computationally inefficient. Modern commercial LLMs may take up terabytes of space and use many compute cycles to execute. In addition, these LLMs can produce inconsistent results; for example, depending on the prior context window, they can exhibit decreased recall with long documents. Nevertheless, it may not be feasible to train a smaller or more efficient LLM from scratch with equivalent accuracy on general tasks. Additionally, training of LLMs is an extremely computationally intensive task, possibly taking several months even running full time on a supercomputer.

Thus, it would be desirable for a system to be able to initially respond to user questions about extracting certain types of answers (e.g., medical questions and related questions) from documents (e.g., sets of clinical notes) using one or more LLMs, train an optimized model from the answers provided by the initial LLM-based system, and then intelligently switch to the optimized model. This may be accomplished by initially sending queries to an LLM, using a heuristic to evaluate when responses are sufficient to properly train another model (e.g., a smaller LLM or another non-LLM model), performing a validation operation to evaluate when the other model has reached a threshold level of performance, and at that point switching over to using the new optimized model in place of the original LLM. In some embodiments, this technique may be used to replace the first LLM (e.g., for evidence extraction) in an LLM system while continuing to use a second LLM for final answer classification. In some embodiments, this technique may also be used to replace the second LLM for final answer classification.

In one embodiment, a method of responding to questions about documents is performed by a computing system. The method includes: (a) initially sending queries to a large language model (LLM), each query including a request for any snippets within a corresponding document that provide evidence in response to a question, the LLM returning a respective response to each query; (b) in response to a heuristic based on the responses returned by the LLM indicating a readiness for optimization, training a new model on the queries and respective responses, the new model being more computationally efficient than the LLM; (c) performing a validation operation on additional responses returned by the new model; and (d) in response to the validation operation indicating that the new model has satisfied a condition, sending future queries to the new model instead of the LLM.

Corresponding apparatuses, systems, and computer program products for performing the method are also provided.

### BRIEF DESCRIPTION OF THE DRAWINGS

The foregoing and other objects, features, and advantages will be apparent from the following description of particular embodiments, as illustrated in the accompanying drawings in which like reference characters refer to the same parts throughout the different views. The drawings are not necessarily to scale, emphasis instead being placed upon illustrating the principles of various embodiments.

FIG. 1 illustrates an example system, apparatus, computer program product, and associated data structures for use in connection with one or more embodiments.

FIG. 2 illustrates an example method in accordance with one or more embodiments.

FIG. 3 illustrates an example interface in accordance with one or more embodiments.

FIG. 4 illustrates an example method in accordance with one or more embodiments.

### DETAILED DESCRIPTION

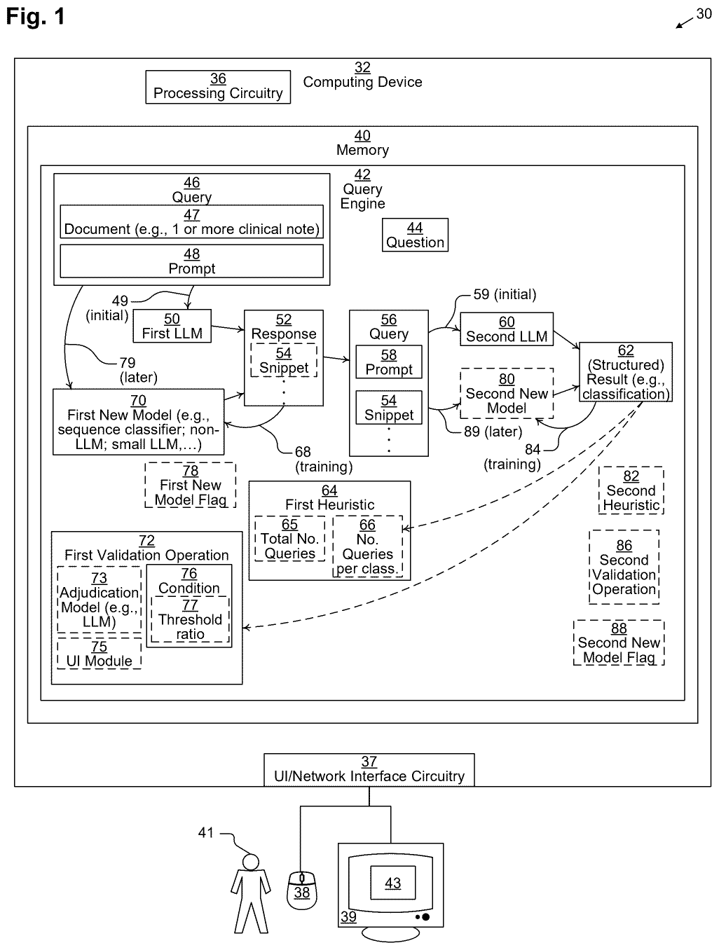

FIG. 1 depicts an example system 30 for use in connection with various embodiments. System 30 includes a computing device 32, one or more input devices 38, one or more display devices 39, and a user 41.

Computing device 32 may be any kind of computing device, such as, for example, a personal computer, laptop, workstation, server, enterprise server, tablet, smartphone, etc. Computing device 32 may include processing circuitry 36, interface circuitry 37 (e.g., user interface (UI) and/or network interface circuitry), and memory 40. Computing device 32 may also include various additional features as is well-known in the art, such as, for example, interconnection buses, etc.

Processing circuitry 36 may include any kind of processor or set of processors configured to perform operations, such as, for example, a microprocessor, a multi-core microprocessor, a digital signal processor, a system on a chip (SoC), a collection of electronic circuits, a similar kind of controller, or any combination of the above.

As depicted in FIG. 1, user 41 directly interfaces with computing device 32 using the one or more input devices 38 and the one or more display devices 39, which are connected via the interface circuitry 37 (e.g., UI circuitry). UI circuitry may include any circuitry needed to communicate with and connect to the one or more input devices 38 and display devices 39. The UI circuitry may include, for example, a keyboard controller, a mouse controller, a touch controller, a serial bus port and controller, a universal serial bus (USB) port and controller, a wireless controller and antenna (e.g., Bluetooth), a graphics adapter and port, etc.

A display device 39 may be any kind of display, including, for example, a CRT screen, LCD screen, LED screen, etc. Input device(s) 38 may include a keyboard, keypad, mouse, trackpad, trackball, pointing stick, joystick, touchscreen (e.g., embedded within display device 39), microphone/voice controller, etc. In some embodiments, instead of being external to computing device 32, the input device 38 and/or display device 39 may be embedded within the computing device 32 (e.g., a cell phone or tablet with an embedded touchscreen). Display device 39 displays a UI 43 to the user 41, and user 41 can enter information into the UI 43 using the one or more input devices 38.

In other embodiments (not depicted), user 41 uses the one or more input devices 38 and display devices 39 to interface with remote UI circuitry (not depicted) on a remote computing device (not depicted) that communicates with the computing device 32 across a network (not depicted). In such a case, the interface circuitry 37 of the computing device 32 may be network interface circuitry, which may include one or more Ethernet cards, cellular modems, Fibre Channel (FC) adapters, InfiniBand adapters, wireless networking adapters (e.g., Wi-Fi), and/or other devices for connecting to a network. The network may be any kind of communications network or set of communications networks, such as, for example, a LAN, WAN, SAN, the Internet, a wireless communication network, a virtual network, a fabric of interconnected switches, etc.

Memory 40 may include any kind of digital system memory, such as, for example, random access memory (RAM). Memory 40 stores an operating system (OS, not depicted, e.g., a Linux, UNIX, Windows, MacOS, or similar operating system), query engine 42, and various drivers and other applications and software modules configured to execute on processing circuitry 36 as well as various data.

In operation, query engine 42 receives a question 44 about a body of text (hereinafter referred to as a “document,” notwithstanding the possibility that the body of text may include several items that are often colloquially called “documents”) 47. For example, in some embodiments, a document 47 may be a set of one or more clinical notes about a patient, and the question 44 may be about an issue of medical relevance, such as whether the patient suffers from a medical condition (and, if so, for more detail about such condition) or about medical test results, etc. Clinical notes may include any kind of electronic records having a plaintext representation, such as, for example, electronic medical records, text files, PDF versions of medical records, images of scanned documents which have been processed using optical character recognition, etc. In other embodiments, document 47 may include another type of information, and the question may relate to a different field of inquiry (e.g., the document 47 may include a set of one or more scouting reports about an athlete, and the question may be a sports-related inquiry, such as whether the athlete has broken any records, whether the athlete has reached base safely in a certain number of consecutive games, etc.). In some embodiments, the question 44 may be received from a user 41, while in other embodiments, the question 44 may come from a database or another source.

Query engine 42 operates to construct a query 46 based on the question 44. For example, the query 46 may include the text of the document 47 (possibly modified through a pre-processing step) and a prompt 48 that restates all or a part of the question 44 in a manner engineered to elicit a proper response from a first large language model (LLM) 50. In an initial mode of operation, query engine 42 sends 49 the query 46 to the first LLM 50, which generates a response 52.

For example, if question 44 is about whether or not a patient has heart disease based on a set of clinical notes making up document 47, the prompt 48 may ask “Does the input set of clinical notes provide evidence that the patient has heart disease? Return all snippets from the input clinical note(s) that support or deny this conclusion.” All snippets 54 or quotations (if any) from the set of clinical notes making up document 47 that support a conclusion of heart disease in the patient would be returned in a response 52 from the first LLM 50.

In some embodiments, query engine 42 operates to construct an additional query 56 based on the question 44 that includes another prompt 58 and a set of all the snippets 54 included within the response 52. In the event that a response 52 contains no snippets 54, no additional query 56 is generated. In some embodiments, additional query 56 may also include additional context from the document 47. In an initial mode of operation, query engine 42 sends 59 the additional query 56 (if any was generated) to a second LLM 60, which generates a result 62. In some embodiments, result 62 is a structured result, such as a classification, for example.

For example, as above, if the question 44 is about whether or not a patient has heart disease based on a set of clinical notes making up document 47, and the response 52 includes at least one snippet 54 relevant to the question of heart disease, the prompt 58 may be “Do the snippets, when analyzed together, imply or strongly suggest that the patient has heart disease? Answer (A) Patient has heart disease, (B) Evidence suggests possible heart disease, (C) Evidence against heart disease, (D) Inconclusive evidence, or (E) No evidence or insufficient evidence. Include quotes to justify the answer.” The output of the second LLM 60 would be a result 62 having a structure that includes one of five classifications A-E, as well as any quotations from the snippets 54 that support that conclusion.

LLMs 50, 60 may be any kind of LLM trained on a large set of training data. In some embodiments, second LLM 60 may be more advanced than first LLM 50 (e.g., it may have more parameters and/or it may be trained on a larger set of data). For example, first LLM 50 may be GPT 3.5 or GPT 3.5 Turbo provided by OpenAI, Inc. of San Francisco, CA, while second LLM 60 may be GPT 4 also provided by OpenAI, Inc.

It should be understood that additional detail about how to implement a system including a question 44 about a document 47, a query 46, a first LLM 50, a response 52 having a set of snippets 54, a second query 56, a second LLM 60, and a result 62 may be found in the U.S. patent application having Ser. No. 18/500,104, entitled “TECHNIQUES FOR REFINING QUERIES TO AN LLM-BASED SYSTEM FOR ANALYZING CLINICAL NOTES” and filed on Nov. 1, 2023, the entire contents and teachings of which are hereby incorporated herein by this reference. It should also be understood that other example configurations may be used instead, such as, for example, configurations making use of only a single query 46 and a single LLM 50 to the exclusion of additional query 56 and second LLM 60.

In continued operation, query engine 42 uses a first heuristic 64 to decide when to begin an optimization procedure via distillation. First heuristic may take various factors into account, such as, for example a total number 65 of queries 46 (or, in some embodiments, additional queries 56) that have been processed by query engine 42 through first LLM 50 (or second LLM 60), a number 66 of structured results 62 that have resulted in each of a plurality of different classifications, etc. For example, in an embodiment, first heuristic 64 only yields an affirmative result once at least 2,000 queries 46 have been processed through first LLM 50 and at least 25 structured results 62 have been returned for each of classifications A-E in the example above.

Once first heuristic 64 returns an affirmative result, query engine 42 begins a training operation 68, which includes using the responses 52 (including any snippets 54 therein) and corresponding queries 46 to construct a training set to train a first new model 70. These responses 52 and queries 46 are queued and saved until the first heuristic 64 initiates the training operation 68.

First new model 70 is a machine learning model that is more computationally efficient than the first LLM 50. First new model 70 may have a different model architecture, fewer parameters, and/or other efficiency optimizations compared to the first LLM 50. In some embodiments, first new model 70 is a sequence classification model (e.g. BERT or GatorTron) trained to directly classify tokens from the text as relevant or irrelevant. Each token is associated with a certain probability of its utility. Sets of tokens above a given probability threshold are then treated as snippets 54. Although first new model 70 may specifically be a non-LLM, in some embodiments, first new model 70 may be an LLM as well, provided that it has a smaller memory footprint, reduced computational complexity, or increased quality compared to the first LLM.

Once the first new model 70 is initially trained, query engine 42 may perform a first validation operation 72 to validate whether the first new model 70 is good enough (yet) to replace the first LLM 50. In some embodiments, first validation operation 72 compares responses 52 generated by first LLM 50 with responses 52 generated by the first new model 70 on a per query 46 basis, achieving validation once a condition 76 is satisfied. In other embodiments, first validation operation 72 compares results 62 generated by second LLM 70 using responses 52 generated by first LLM 50 with results 62 generated by second LLM 70 using responses 52 generated by the first new model 70 on a per query 56 basis, achieving validation once the condition 76 is satisfied.

Depending on the embodiment, the comparison of the first validation operation 72 may be performed by a user 41 via a UI 75 module of the first validation operation 72 and displayed as UI 43 in display 39 or by an adjudication model 73. Adjudication model 73 may be any kind of machine learning model capable of predicting which results are of higher quality, such as an LLM (e.g., GPT 4).

In an embodiment, condition 76 represents a ratio of responses 52 (or results 62) based on the first new model 70 that are deemed superior to the corresponding responses 52 (or results 62) based on the first LLM 50 over responses 52 (or results 62) based on the first LLM 50 that are deemed superior to the corresponding responses 52 (or results 62) based on the first new model 70 exceeding a threshold value 77 (e.g., 1:1). Once the condition 76 is satisfied, validation is achieved, and query engine 42 subsequently sends 79 queries 46 for future questions 44 to the first new model 70 instead of sending those queries 46 to the first LLM 50. In some embodiments, this may be accomplished by setting a first new model flag 78; query engine 42 checks whether the first new model flag 78 is set upon generating each query 46, sending the query 46 to the first LLM 50 when the first new model flag 78 has not yet been set and to the first new model 70 once the first new model flag 78 has been set.

In some embodiments, a similar process is followed to distill the second LLM 60 into a second new model 80, the second new model 80 similarly being a machine learning model that is more computationally efficient than the second LLM 60. Second new model 80 is similarly smaller and more computationally efficient than second LLM 60.

Once a second heuristic 82 (which may be similar to or different than first heuristic 64) returns an affirmative result, query engine 42 begins a training operation 84, which includes sending the results 62 and corresponding queries 56 to the second new model 80 to be trained. Once the second new model 80 is initially trained, query engine 42 may similarly perform a second validation operation 86 (which may be similar to or different than first validation operation 72) to validate whether the second new model 80 is good enough (yet) to replace the second LLM 60, and query engine 42 may make use of a second new model flag 88 similar to first new model flag 78.

Memory 40 may also store various other data structures used by the OS, query engine 42, first LLM 50, second LLM 60, first heuristic 64, first new model 70, first validation operation 72, second heuristic 82, second new model 80, second validation operation 86, and/or various other applications and drivers. In some embodiments, memory 40 may also include a persistent storage portion. Persistent storage portion of memory 40 may be made up of one or more persistent storage devices, such as, for example, magnetic disks, flash drives, solid-state storage drives, or other types of storage drives. Persistent storage portion of memory 40 is configured to store programs and data even while the computing device 32 is powered off. The OS, query engine 42, first LLM 50, second LLM 60, first heuristic 64, first new model 70, first validation operation 72, second heuristic 82, second new model 80, second validation operation 86, and/or various other applications and drivers are typically stored in this persistent storage portion of memory 40 so that they may be loaded into a system portion of memory 40 upon a system restart or as needed. The OS, query engine 42, first LLM 50, second LLM 60, first heuristic 64, first new model 70, first validation operation 72, second heuristic 82, second new model 80, second validation operation 86, and/or various other applications and drivers, when stored in non-transitory form either in the volatile or persistent portion of memory 40 (which may be referred to as a non-transitory computer-readable storage medium), each form a computer program product. The processing circuitry 36 running one or more applications thus forms a specialized circuit constructed and arranged to carry out the various processes described herein.

In some embodiments (not depicted), instead of the above-described functions of computing device 32 being performed entirely by processing circuitry 36 of a single computing device 32 with corresponding data stored entirely within memory 40 of computing device 32, the functions and data may be distributed across several computing devices communicatively coupled via a network.

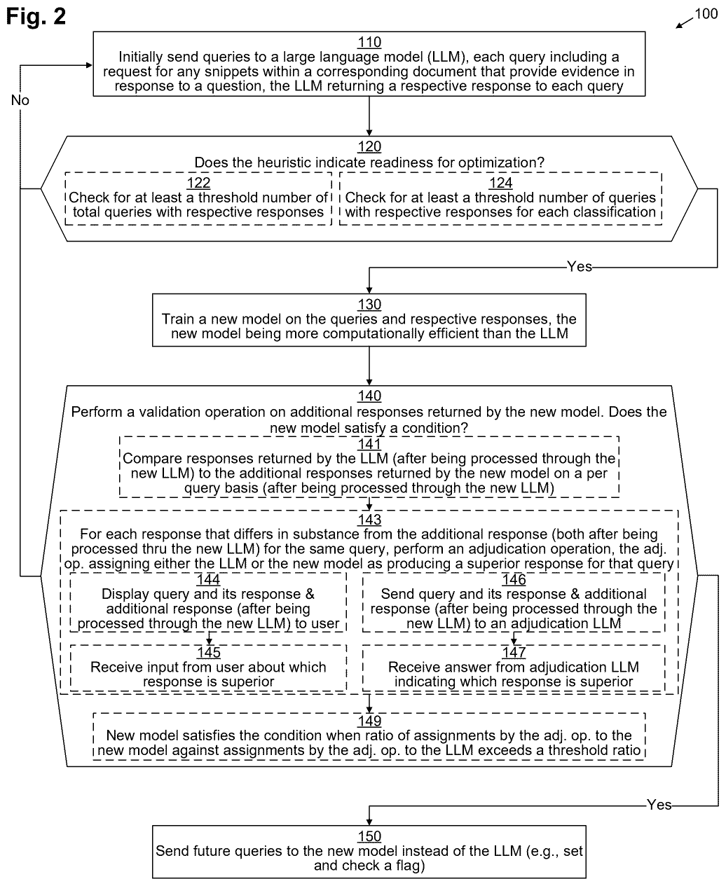

FIG. 2 illustrates an example method 100 performed by a system 30 for responding to questions 44 about documents 47. It should be understood that any time a piece of software (e.g., OS, query engine 42, first LLM 50, second LLM 60, first heuristic 64, first new model 70, first validation operation 72, second heuristic 82, second new model 80, second validation operation 86, etc.) is described as performing a method, process, step, or function, what is meant is that a computing device 32 on which that piece of software is running performs the method, process, step, or function when executing that piece of software on its processing circuitry 36. It should be understood that one or more of the steps or sub-steps of method 100 may be omitted in some embodiments. Similarly, in some embodiments, one or more steps or sub-steps may be combined together or performed in a different order.

It should be understood that although method 100 is described primarily in the context of operating and training first LLM 50 and first new model 70, it can also be used in the context of operating and training second LLM 60 and second new model 80. Such secondary use will be mentioned in square brackets, [like so].

In step 110, query engine 42 initially 49 [or 59] sends queries 46 [or additional queries 56] to first LLM 50 [or second LLM 60]. Each query 46 includes a request (e.g., within a prompt 48) for any snippets 54 within a corresponding document 47 that provide evidence in response to a question 44. [Each additional query 56 includes a prompt 58 and one or more snippets 54, and, in some embodiments, additional context from document 47.] In response to each such query 46 [56], the first LLM 50 [second LLM 60] returns a respective response 52 [result 62]. In some embodiments, during this initial stage (step 110), each query 46 and its corresponding response 52 [result 62] is stored for later use in step 130, below.

In step 120, query engine 42 operates first heuristic 64 [or second heuristic 82] to determine if the heuristic indicates readiness for optimization. In some embodiments, in sub-step 122, first heuristic 64 [second heuristic 82] checks to ensure that at least a threshold number (e.g., 2,000) of queries 46 have been processed through first LLM 50 [second LLM 60]. In some embodiments, in sub-step 124, first heuristic 64 [second heuristic 82] checks to ensure that at least a threshold number (e.g., 25) structured results 62 have been returned for each of a plurality of classifications that have been defined. If first heuristic 64 [second heuristic 82] yields a negative result, then operation returns back to step 110; otherwise, operation proceeds with step 130.

In some embodiments, step 120 is performed separately for different classes of questions 44. For example, a first class (class 1) of questions may ask about whether a patient has a disease (e.g., heart disease), providing five classifications (A) Patient has X disease, (B) Evidence suggests possible X disease, (C) Evidence against X disease, (D) Inconclusive evidence, or (E) No evidence or insufficient evidence. A second class (class 2) of questions may ask about whether a set of clinical notes indicates that a patient has a test result (e.g., creatinine) level greater than (or some other comparator, such as less than, equal to, etc.) a threshold, with classifications (I) “yes,” (II) “insufficient evidence,” (III) “lacks mention,” and (IV) “explicit no.” The first heuristic 64 [or second heuristic 82] is evaluated separately for each such class of question.

In step 130, query engine 42 trains 68 first new model 70 [or second new model 80] on the queries 46 (for the particular class of questions for which step 120 produced the affirmative answer) and their respective responses 52 [results 62] that were stored in step 110 above. First new model 70 [second new model 80] is more computationally efficient than first LLM 60 [second new model 80]. This training 68 may include tuning the first new model 70 [second new model 80] and choosing relevant hyperparameters for it to use.

Once the first new model 70 [or second new model 80] has been trained (for a particular class of questions 44), query engine 42 performs first validation operation 72 [or second validation operation 86] on additional responses 52 [results 62] that are returned by the first new model 70 [second new model 80] (for that class of questions 44), checking whether the first new model 70 [second new model 80] satisfies a condition 76 (for that class of questions 44). If the first new model 70 [second new model 80] satisfies the condition 76, then operation proceeds with step 150, in which query engine 42 subsequently 79 sends future queries 46 [additional queries 56] (for that class of questions 44) to first new model 70 [second new model 80] instead of first LLM 50 [second LLM 60]. Step 150 may include setting first new flag 78 [second new flag 88] (for that class of questions 44), and then checking the value of the first new flag 78 [second new flag 88] (for that class of questions 44) for each new query 46 [56]. It should be understood that step 110 may also include checking the value of the first new flag 78 [second new flag 88] (for that class of questions 44) for each query 46 [56].

In some embodiments, step 140 may include sub-steps 141, 143, and 149, described only in the context of first validation operation 72, although similar sub-steps may also be performed in the context of second validation operation 86. In sub-step 141, first validation operation 72 compares responses 52 returned by the first LLM 50 to the additional responses 52 returned by the first new model 70 on a per query 46 basis. In some embodiments (as described below), instead of directly comparing responses 52, the responses 52 are compared indirectly after having been processed through the second LLM 60. Thus, for example, a first query 46(1) that was sent to first LLM 50 yields a response 52(1)(a), which is then sent to second LLM 60, yielding result 62(1)(a). Similarly, the same first query 46(1) is also sent to first new model 70, yielding a response 52(1)(b), which is then sent to second LLM 60, yielding result 62(1)(b). In the same manner a second query 46(2) that was sent to first LLM 50 ultimately yields a result 62(2)(a), and the same second query 46(2) that was sent to first new model 70 ultimately yields a result 62(2)(b). Then, in sub-step 141, result 62(1)(a) is compared to result 62(1)(b), and result 62(2)(a) is compared to result 62(2)(b). In some cases (for example, in the comparison of results 62(1)(a), 62(1)(b)), the results are identical. These identical results may be ignored for the purposes of sub-step 143.

In sub-step 143, for results 62(X)(a), 62(X)(b) that are not identical for a particular query 46(X) (e.g., results 62(2)(a), 62(2)(b)), first validation operation 72 performs an adjudication operation that either assigns the first LLM 50 or the first new model 70 as producing a superior result 62 (i.e., if result 62(2)(a) is superior to result 62(2)(b)), then the adjudication operation assigns the first LLM 50 as being superior; if result 62(2)(b) is superior to result 62(2)(a)), then the adjudication operation assigns the first new model 70 as being superior). In some embodiments, sub-step 143 may include sub-steps 144-145, while in other embodiments, sub-step 143 may include sub-steps 146-147.

In sub-step 144, UI module 75 displays the query 46(X) or question 44(X) and its corresponding result 62(X)(a) and additional result 62(X)(b) (or, in an alternative embodiment, its corresponding response 52(X)(a) and additional response 52(X)(b)) to the user 41 within UI 43 on display 39.

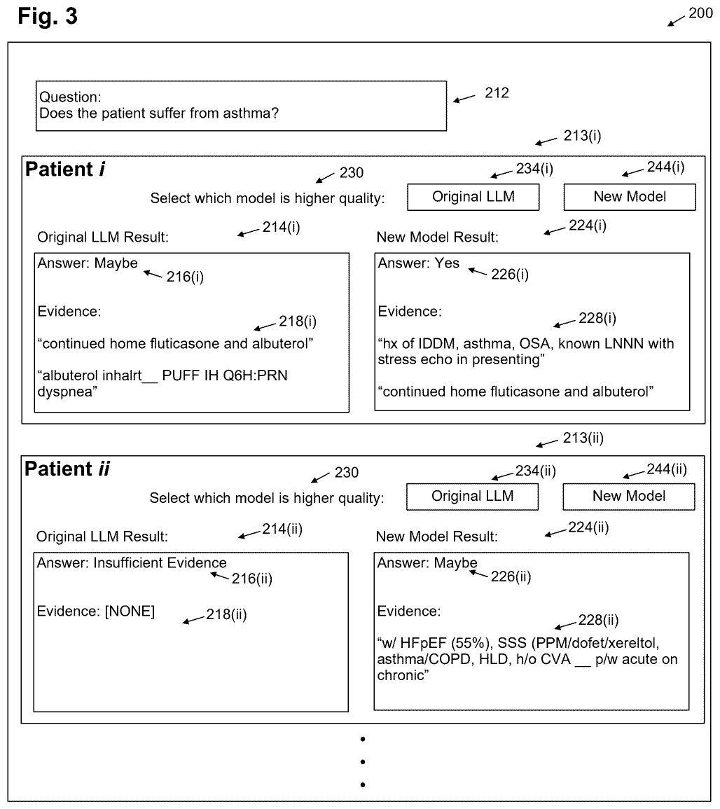

FIG. 3 depicts an example UI page 200 to be displayed within UI 43 in the context of sub-step 144. As depicted in FIG. 3, question 44(X) is shown in question box 212 (“Does the patient suffer from asthma?”). Result boxes 213 (depicted as result boxes 213(i), 213(ii), . . . , corresponding to different patients i, ii, . . . ) each display a depiction 216 of result 62(X)(a) as well as indications 218 of snippets 54 identified by response 52(X)(a) within an original LLM result box 214. A new model response box 224 displays a depiction 226 of result 62(X)(b) as well as indications 228 of snippets 54 identified within response 52(X)(b). Each patient i, ii, . . . has a corresponding document 47 (e.g., a set of clinical notes for that patient). As can be seen in result box 213(i) for patient i, although there is some overlap between the indications 218(i), 228(i), there is some disagreement between them, illustrating a difference between the performance of first LLM 50 and first new model 70. Due, in part to that difference, the depictions 216(i), 226(i) of the results 62(X)(a), 62(X)(b) for patient i also differ, with original LLM result 214(i) indicating “Maybe” and new model result 224(i) indicating “Yes.” As can be seen in result box 213(ii) for patient ii, indication 218(ii) shows that there were no snippets 54 provided by first LLM 50, while indication 228(ii) shows a single snippet 54 provided by new model 70. Due, in part to that difference, the depictions 216(ii), 226(ii) of the results 62(X)(a), 62(X)(b) for patient ii also differ, with original LLM result 214(ii) indicating “Insufficient Evidence” and new model result 224(ii) indicating “Maybe.”

Returning to FIG. 2, in sub-step 145, UI module 75 received input from the user 41 via UI 43. FIG. 3 depicts a request 230 for the user 41 to select button 234 if response box 214 (representing the output of first LLM 50) is more accurate or button 244 if response box 224 (representing the output of first new model 70) is more accurate. Thus, when user 41 operates input device 38 to select one of the buttons 234(i), 244(i), the superior model 50, 70 is decided for question 44(X) as applied to the document 47 (e.g., clinical notes) for patient i; and when user 41 operates input device 38 to select one of the buttons 234(ii), 244(ii), the superior model 50, 70 is decided for question 44(X) as applied to the document 47 (e.g., clinical notes) for patient ii.

In sub-step 146, first validation operation 72 sends the query 46(X) or question 44(X) and its corresponding result 62(X)(a) and additional result 62(X)(b) (or, in an alternative embodiment, its corresponding response 52(X)(a) and additional response 52(X)(b)) to adjudication model 73 with a request to decide which is superior. In sub-step 147, first validation operation 72 receives an answer from the adjudication model 73, deciding which of the models 50, 70 is superior for question 44(X), query 46(X).

In another embodiment (not depicted), sub-step 143 may instead be performed with reference to a set of labels (not depicted) that identify the correct responses 52 and/or results 62 for each query 46. In such embodiments, the correct responses 52 and/or results 62 may have been determined by an objective source of truth in advance.

After sub-step 143 (i.e., either sub-steps 144-145, 146-147, or the undepicted embodiment) is performed for each query 46(X) or question 44(X) whose respective results 62 are not identical, sub-step 149 is performed. In sub-step 149, first validation operation 72 decides that the new model 70 has satisfied the condition 76 once the ratio of adjudications in favor of the first new model 70 against adjudications in favor of the first LLM 50 exceeds the threshold ratio 77.

On one example task to identify patients who have taken or are currently taking chemotherapy, there were 1009 documents 47 and 43 differences. Of those 43 differences, 35 were ruled in favor of the first new model 70, and 8 were ruled in favor of the first LLM 50. This yielded a precision/recall of 0.988 and 0.967 for the first new model 70, and 0.855 and 0.945 for the first LLM 50. On another example task of assessing the extent of resection of brain tumors, there was 86% concordance between the first new model 70 and the first LLM 50, with approximately comparable performance after adjudication.

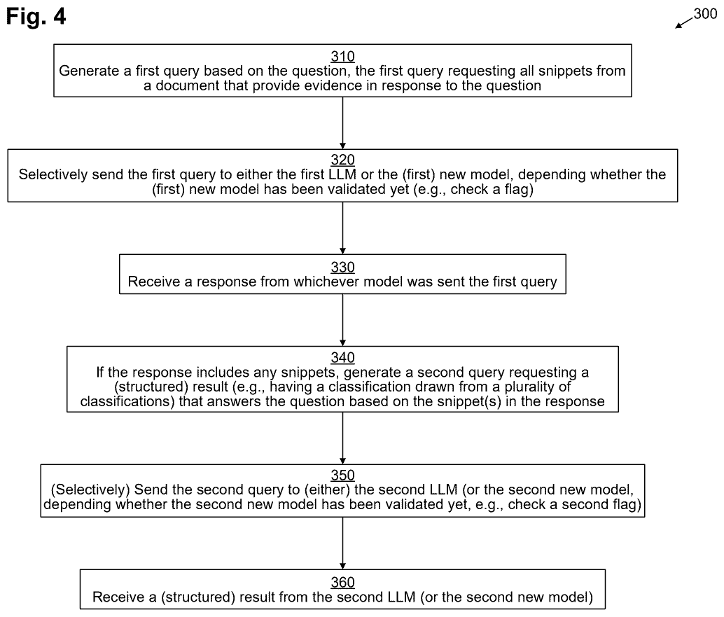

FIG. 4 depicts an example method 300 for responding to a question 44 in accordance with some embodiments.

In step 310, query engine 42 generates a first query 46 based on the question 44, the first query 46 requesting (via a prompt 48) all snippets 54 from a document 47 that provide evidence relevant to responding to the question 44. An example question 44(X) is “Does the patient in this note have heart disease, and, if so, what is the etiology? Yes/No/Maybe. Yes/Maybe: Ischemic/COPD/Hypertensive heart disease/Alcohol/Other.” An example prompt 48(X) of the query 46(X) for that question 44(X) is “Please return all direct quotes from the note that are relevant to the patient's heart failure etiology (if any).” In some embodiments, the prompt 48 may also include one or more examples to guide the first LLM 50 or first new model 70 in preparing its response. Thus, one such example might provide the full text of a different clinical note as well as a set of quotes drawn from that note that are relevant to the question 44.

In step 320, query engine 42 selectively sends the first query 46 to either the first LLM 50 (step 49 from FIG. 1) or the first new model 70 (step 79 from FIG. 1), depending whether the first new model 70 has yet been validated by the first validation operation 72. In an embodiment, step 320 takes into account a class of the question 44. Thus, for example, if the question is in class 1 (see discussion of step 120 above), query engine 42 selectively sends the first query 46 to either the first LLM 50 (step 49 from FIG. 1) or the first new model 70 (step 79 from FIG. 1), depending whether the first new model 70 has yet been validated by the first validation operation 72 for class 1; and if the question is in class 2 (see discussion of step 120 above), query engine 42 selectively sends the first query 46 to either the first LLM 50 (step 49 from FIG. 1) or the first new model 70 (step 79 from FIG. 1), depending whether the first new model 70 has yet been validated by the first validation operation 72 for class 2. In some embodiments, step 320 is performed with reference to a first new model flag 78, which, in some embodiments, may be set on a per class basis. In some embodiments (not depicted), the flag 78 may be ignored if a document 47 has unusual characteristics according to another heuristic (e.g., it is much shorter or much longer than the typical documents 47 that have been processed), causing the first query 46 to be sent to the first LLM 50 even if the flag 78 indicates that it should be sent to the first new model 70.

In step 330, query engine 42 receives a response 52 from whichever model 50, 70 was sent the query 46 in step 320.

In step 340, if the response 52 includes at least one snippet 54, query engine 42 generates a second query 56 that requests (via a prompt 58) a result 62 that answers the question 44 based on the one or more snippet(s) 54 returned within the response 52. In some embodiments, additional query 56 may also include additional context from the document 47. In some embodiments, prompt 58 provides a structure for the response 62, such as a set of possible classifications, for example. An example prompt 58(X) for a query 56(X) to answer question 46(X) is “Based on these snippets, does the patient have heart disease, and, if so, what is the etiology? Yes/No/Maybe. If Yes or Maybe, possible etiologies are: Ischemic/COPD/Hypertensive heart disease/Alcohol/Other.”

In step 350, query engine 42 sends the second query 56 to a model 60, 80 for an answer. In some embodiments, query engine 42 always sends the second query 56 to second LLM 60. In other embodiments, query engine 42 selectively sends the second query 56 to either the second LLM 60 (step 59 from FIG. 1) or the second new model 80 (step 89 from FIG. 1), depending whether the second new model 80 has yet been validated by the second validation operation 86. In an embodiment, step 350 takes into account a class of the question 44 in making this selection. In some embodiments, step 350 is performed with reference to a second new model flag 88, which, in some embodiments, may be set on a per class basis. In some embodiments (not depicted), the flag 88 may be ignored if either the document 47 or the snippets 54 produced has unusual characteristics according to another heuristic (e.g., a very large number of snippets 54 has been produced within response 52), causing the second query 56 to be sent to the second LLM 60 even if the flag 78 indicates that it should be sent to the second new model 80.

In step 360, query engine 42 receives a result 62 (e.g., a structured result) from whichever model 60, 80 was sent the query 56 in step 350. This result 62 may be displayed to the user 41 within UI 43, for example. As another example, the result 62 may be stored within a database for later analysis.

While various embodiments of the invention have been particularly shown and described, it will be understood by those skilled in the art that various changes in form and details may be made therein without departing from the spirit and scope of the invention as defined by the appended claims.

It should be understood that although various embodiments have been described as being methods, software embodying these methods is also included. Thus, one embodiment includes at least one tangible computer-readable medium (such as, for example, a hard disk, a floppy disk, an optical disk, computer memory, flash memory, etc.) programmed with instructions, which, when performed by a computer or a set of computers, cause one or more of the methods described in various embodiments to be performed. Another embodiment includes a computer which is programmed to perform one or more of the methods described in various embodiments.

Furthermore, it should be understood that all embodiments which have been described may be combined in all possible combinations with each other, except to the extent that such combinations have been explicitly excluded.

Finally, nothing in this Specification shall be construed as an admission of any sort. Even if a technique, method, apparatus, or other concept is specifically labeled as “background” or as “conventional,” Applicants make no admission that such technique, method, apparatus, or other concept is actually prior art under 35 U.S.C. § 102 or 103, such determination being a legal determination that depends upon many factors, not all of which are known to Applicants at this time.

What is claimed is:

1. A method performed by a computing system of responding to questions about documents, the method comprising:
initially sending queries to a large language model (LLM), each query including a request for any snippets within a corresponding document that provide evidence in response to a question, the LLM returning a respective response to each query;
in response to a heuristic based on the responses returned by the LLM indicating a readiness for optimization, training a new model on the queries and respective responses, the new model being more computationally efficient than the LLM;
performing a validation operation on additional responses returned by the new model; and
in response to the validation operation indicating that the new model has satisfied a condition, sending future queries to the new model instead of the LLM.

2. The method of claim 1 wherein the heuristic ensures that there are at least a threshold number of queries with respective responses prior to indicating readiness for optimization.

3. The method of claim 2 wherein:
each response having one or more snippets is sent to a second LLM requesting a determination of a classification of a plurality of different classifications, the determined classification answering the question based on the one or more snippets; and
the heuristic further ensures that each classification of the plurality of different classifications has been determined at least a threshold number of times prior to indicating readiness for optimization.

4. The method of claim 1 wherein:
each response having one or more snippets is sent to a second LLM requesting a determination of a classification of a plurality of different classifications, the determined classification answering the question based on the one or more snippets; and
the heuristic ensures that each classification of the plurality of different classifications has been determined at least a threshold number of times prior to indicating readiness for optimization.

5. The method of claim 1 wherein:
the method further comprises sending each response having one or more snippets and each additional response having one or more snippets to another LLM, the other LLM returning a respective structured result in response, each returned structured result answering the question based on the corresponding one or more snippets;
performing the validation operation includes:
comparing(i) the structured results (hereinafter the initial structured results) returned by the other LLM in response to the responses to(ii) the structured results (hereinafter the new structured results) returned by the other LLM in response to the additional responses on a per query basis; and
for each initial structured result that differs in substance from the new structured result for the same query, performing an adjudication operation, the adjudication operation assigning either the LLM or the new model as producing a superior response for that query; and
the validation operation indicates that the new model has satisfied the condition when a ratio of assignments by the adjudication operation to the new model against assignments by the adjudication operation to the LLM exceeds a threshold ratio.

6. The method of claim 5 wherein performing the adjudication operation for a query includes:
displaying that query and its corresponding initial structured result and new structured result to a user; and
receiving an indication from the user of whether the initial structured result or the new structured result is superior.

7. The method of claim 5 wherein performing the adjudication operation for a query includes:
sending that query and its corresponding initial structured result and new structured result to another LLM; and
receiving an indication from the other LLM of whether the initial structured result or the new structured result is superior.

8. The method of claim 1 wherein the new model is a sequence classification model.

9. The method of claim 1 wherein the method further comprises, in response to the validation operation indicating that the new model has not satisfied the condition, continuing to send future queries to the LLM until the validation operation indicates that the new model has satisfied the condition.

10. The method of claim 1 wherein each query relates to a medical question and each corresponding document includes one or more medical notes.

11. The method of claim 1 wherein the method further comprises:
initially sending each response having one or more snippets to another LLM requesting a determination of a structured result, the determined structured result answering the question based on the one or more snippets;
in response to another heuristic based on the structured results returned by the other LLM indicating a readiness for optimization, training another new model on the responses, the other new model being more computationally efficient than the other LLM;
performing another validation operation on additional structured results returned by the other new model; and
in response to the validation operation indicating that the other new model has satisfied another condition, sending future responses to the other new model instead of the other LLM.

12. A computer program product comprising a non-transitory computer-readable storage medium storing instructions, which when performed by processing circuitry of a computing system, cause the computing system to respond to questions about documents by:
initially sending queries to a large language model (LLM), each query including a request for any snippets within a corresponding document that provide evidence in response to a question, the LLM returning a respective response to each query;
in response to a heuristic based on the responses returned by the LLM indicating a readiness for optimization, training a new model on the queries and respective responses, the new model being more computationally efficient than the LLM;
performing a validation operation on additional responses returned by the new model; and
in response to the validation operation indicating that the new model has satisfied a condition, sending future queries to the new model instead of the LLM.

13. The computer program product of claim 12 wherein the heuristic ensures that there are at least a threshold number of queries with respective responses prior to indicating readiness for optimization.

14. The computer program product of claim 13 wherein:
each response having one or more snippets is sent to a second LLM requesting a determination of a classification of a plurality of different classifications, the determined classification answering the question based on the one or more snippets; and
the heuristic further ensures that each classification of the plurality of different classifications has been determined at least a threshold number of times prior to indicating readiness for optimization.

15. The computer program product of claim 12 wherein:
each response having one or more snippets is sent to a second LLM requesting a determination of a classification of a plurality of different classifications, the determined classification answering the question based on the one or more snippets; and
the heuristic ensures that each classification of the plurality of different classifications has been determined at least a threshold number of times prior to indicating readiness for optimization.

16. The computer program product of claim 12 wherein:
the instructions, when performed by the processing circuitry, further cause the computing system to send each response having one or more snippets and each additional response having one or more snippets to another LLM, the other LLM returning a respective structured result in response, each returned structured result answering the question based on the corresponding one or more snippets;
performing the validation operation includes:
comparing(i) the structured results (hereinafter the initial structured results) returned by the other LLM in response to the responses to(ii) the structured results (hereinafter the new structured results) returned by the other LLM in response to the additional responses on a per query basis; and
for each initial structured result that differs in substance from the new structured result for the same query, performing an adjudication operation, the adjudication operation assigning either the LLM or the new model as producing a superior response for that query; and
the validation operation indicates that the new model has satisfied the condition when a ratio of assignments by the adjudication operation to the new model against assignments by the adjudication operation to the LLM exceeds a threshold ratio.

17. The computer program product of claim 16 wherein performing the adjudication operation for a query includes:
displaying that query and its corresponding initial structured result and new structured result to a user; and
receiving an indication from the user of whether the initial structured result or the new structured result is superior.

18. The computer program product of claim 16 wherein performing the adjudication operation for a query includes:
sending that query and its corresponding initial structured result and new structured result to another LLM; and
receiving an indication from the other LLM of whether the initial structured result or the new structured result is superior.

19. The computer program product of claim 12 wherein the new model is a sequence classification model.

20. A computing system comprising:
interface circuitry; and
processing circuitry coupled to memory configured to respond to questions about documents by:
initially sending queries to a large language model (LLM), each query including a request for any snippets within a corresponding document that provide evidence in response to a question, the LLM returning a respective response to each query;
in response to a heuristic based on the responses returned by the LLM indicating a readiness for optimization, training a new model on the queries and respective responses, the new model being more computationally efficient than the LLM;
performing a validation operation on additional responses returned by the new model; and
in response to the validation operation indicating that the new model has satisfied a condition, sending future queries to the new model instead of the LLM.
